(19) United States
(12) Patent Application Publication
(10) Pub. No.: US 2022/0236147 A1
(43) Pub. Date: Jul. 28, 2022

(54) RESPIRATORY DISEASE SURVEILLANCE SYSTEMS AND METHODS USING HIGH FLOWRATE AEROSOL CAPTURE FOR RAPID ON-SITE ANALYSIS

(71) Applicant: Zeteo Tech, Inc., Sykesville, MD (US)

(72) Inventors: Charles J. Call, Albuquerque, NM (US); George Bajszar, Albuquerque, NM (US); Patrick Call, Albuquerque, NM (US)

(73) Assignee: Zeteo Tech, Inc., Sykesville, MD (US)

(21) Appl. No.: 17/586,679

(22) Filed: Jan. 27, 2022

Related U.S. Application Data
(60) Provisional application No. 63/142,482, filed on Jan. 27, 2021.
(60) Provisional application No. 63/303,438, filed on Jan. 26, 2022.

(51) Int. Cl. G01N 1/22; C12Q 1/70; C12Q 1/6806; C12Q 1/689

(52) U.S. Cl. CPC G01N 1/2273; C12Q 1/689; C12Q 1/6806; C12Q 1/70

(57) ABSTRACT

Disclosed are methods and systems for analyzing exhaled breath aerosol particles present in the ambient environment using high flowrate aerosol collector systems and sensitive and specific nucleic acid analysis systems to determine if active spreaders of a respiratory disease are present in an indoor space. The disclosed systems and methods provide for a diagnostic test result in less than about 30 minutes.

### RELATED APPLICATIONS

This application is related to and claims the benefit of U.S. Provisional Application 63/142,482, filed Jan. 27, 2021, and titled “Diagnostic Systems and Methods Using High Flow Rate Aerosol Capture for On-site Analysis,” and U.S. Provisional Application 63/303,438, filed Jan. 26, 2022, and titled “Respiratory Disease Surveillance Systems and Methods Using High Flow Rate Aerosol Capture for Rapid On-site Analysis,” which are both hereby incorporated by reference in each of their entireties.

### FEDERALLY SPONSORED RESEARCH AND DEVELOPMENT

None.

### FIELD

This disclosure relates to methods and devices for analyzing particles in an indoor environment using various aerosol sampling and diagnostic tools to enable rapid and low-cost methods for Active Case Finding (ACF) in occupied indoor spaces. More particularly, but not by way of limitation, the present disclosure relates to methods and devices for high air flowrate collection of exhaled breath aerosols present in indoor air combined with highly sensitive and specific on-site genomic analysis suitable for ACF of diseases such as COVID-19 from ambient air samples.

### BACKGROUND

Coronavirus Disease (COVID-19) is a disease caused by the newly emerged coronavirus SARS-CoV-2. This new coronavirus is a respiratory virus and spreads primarily through droplets generated when an infected person coughs or sneezes, or through droplets of saliva or discharge from the nose. The novel coronavirus is highly contagious and has created an ongoing COVID-19 pandemic, which suggests that this virus is spreading more rapidly than influenza. To help in mitigation, rapid collection and detection devices and methods are needed.

The best method to control transmission of COVID-19 and similar respiratory infections transmitted by aerosol is to promptly identify active spreaders of the pathogen and place them in isolation from the general population. The state-of-the-art method for ACF of COVID-19 patients is to collect a nasal swab or saliva sample from as many members of the local population as often as possible, which are then analyzed at a laboratory off-site. The problem is that this requires an extraordinary number of tests, trained personnel for collecting samples, laboratory analysis equipment and trained laboratory technicians. Turn-around times from sample collection to analysis result is often as much as 24 hours, and frequently as much as 48-72 hours. Furthermore, only a small fraction of the local population is tested, which requires time consuming contact tracing and isolation to mitigate spread of the virus.

Methods and systems to rapidly determine if a COVID-19 spreader (infected person) is in a defined indoor space is urgently needed to isolate that person and contain the spread of the COVID-19 pandemic. Relative to frequent testing with nasal swabs and saliva samples from each individual, new methods and systems should be easier to implement and less invasive, have a shorter analysis time, consume fewer disposable assays, be amenable to on-site field use, and be less labor intensive to implement.

Exhaled breath aerosols (“EBA”) in ambient air can be collected and concentrated into an aqueous liquid “hydrosol” sample. For example, U.S. Pat. No. 6,729,196 titled “BIOLOGICAL INDIVIDUAL SAMPLER,” discloses a portable sampling unit capable of separating particulates, including biological organisms, from air. The unit, which is typically, a battery-powered portable unit collects particles using a rotating impactor that captures particles on a dry surface. The surfaces are then rinsed with a buffer solution to collect a liquid sample comprising the collected aerosol particles in a collection vial. A combined particle impact collector and fan is used to both move fluid through the sampling unit and to collect particulates. The combined particle impact collector may be a disposable unit that is removable and could be replaced with a fresh unit after each sampling period. The disposable unit is placed in a rinse station, where a liquid sample is extracted for later analysis. Alternatively, a disposable detection unit is incorporated in the sampling unit to provide real time detection of chemical toxins and/or biological pathogens. Preferably, the detector unit includes micro-fluidic channels so that a minimum amount of sample and test reagents are required. The combined impact collector may be integral to the sampling unit, rather than a separate disposable item. In this case, the combined particle collector and fan is rinsed in the unit and the liquid sample is collected. Air flow rate is fixed at about 150-200 liters/min (L/min) which limits the viability of the disclosed sampler for quickly sampling ambient air in large indoor spaces. After rinsing, the sampler yields about 2 to 7 ml of liquid sample. Sample collection time could range from about 5 min to about 30 min. Size of particles collected could range from about 1 micron to about 10 micron. Cross-contamination of samples is an issue with this type of aerosol collectors, which requires the sampler internal surfaces to be cleaned using cleaning fluids after each sample collection. Further, the unit generates significant noise while running and does not support silent and/or non-obtrusive operating requirements for air sampling in ambient air and air inside office buildings, airports and other infrastructure. A typical collection efficiency is 75% to 80% for particles greater than 2 microns in size, where collection efficiency (or concentration factor) is the ratio of the number of aerosol particles that are collected in the liquid sample to the number of particles that enter the collector system.

SARS-CoV-2 virus may be identified in EBA by culture, nucleic acid amplification technologies (NAAT) such as polymerase chain reaction (PCR), isothermal nucleic acid amplification, and immunoassay technique such as ELISA. NAATs for SARS-CoV-2 specifically identify the RNA (ribonucleic acid) sequences that comprise the genetic material of the virus. Among these techniques, reverse transcriptase PCR or RT-PCR, has been proven to be rapid (outputting a result in less than about 1 hour), highly sensitive and highly specific to an RNA virus such as SARS-CoV-2. Other types of assays that use RNA amplification may also be suitable. Mass spectrometric (MS) techniques such as matrix assisted laser desorption ionization time-of-flight MS (MALDI-TOFMS) and antibody-based assays such as ELISA and lateral flow assays may also be sensitive and specific.

Further, the time associated with a diagnostic assay is a critical parameter for a PON (“Point of Need”) test. ACF is an example of a field diagnostic assay because, by definition, ACF takes place outside the healthcare system. In the U.S., a POC (“Point of Care”) test should provide an answer in 20 minutes or less. If not, the test may be considered to be too slow and not acceptable for achieving short patient wait-times. In the developing world, and especially in countries with a history of tuberculosis (TB) prevalence, the GeneXpert (Cepheid, Inc., Sunnyvale, Calif.) and FilmArray (BioFire Diagnostics, Inc., Salt Lake City, Utah) products may be used to provide diagnosis in about 45 minutes (“min.”). The GeneXpert Ultra is a genomics-based point of need (POC) diagnostic device which uses PCR technology. Another PCR device is the FilmArray™ (BioFire Diagnostics, Inc., Salt Lake City, Utah).

Any of these NAAT devices may be used in combination with a high-flow-rate environmental collection device and method to perform ACF of COVID-19 and other respiratory diseases on-site, or at the “point of need” (PON). Either device may be integrated with a system that samples air to analyze air samples for airborne pathogens. The BDS system (Northup Grumman, Edgewood, Md.), is being used for screening U.S. Postal Service mail for bacterial spores that cause anthrax as the mail passes through distribution centers. It combines a wetted-wall cyclone with a GeneXpert PCR system to autonomously sample air and report if pathogens are present.

PON-NAAT assay devices have a relatively high cost-per-test and take approximately up to an hour to sample, complete the assay and provide a result. In general, PCR-based diagnostics are not ideal for screening for PON-ACF applications due to both the extended time needed for sampling and analysis, and the relatively high cost per test. However, with advances in technology, and due to the extraordinary and global economic and public health impact of the COVID-19 pandemic, the economics and need have shifted to the point where these PON devices, combined with specific methods of sample collection, may be used to address the need for ACF of COVID-19 spreaders.

PON-ACF devices and methods should be capable of yielding high sensitivity and specificity. Sensitivity is generally the ability of a test or test method to correctly identify patients with a disease. Specificity is the ability of a test or test method to correctly identify people without the disease. The sensitivity of a test or assay may be calculated as the number of true positives as a fraction of the sum of measured number of true positives and the number of false negatives. Stated differently, the sensitivity of a test is the number of measured positives divided by the actual number of true positives if the test was accurate 100% of the time. Specificity may be calculated as the number of negative test results as a fraction of the sum of number of true negatives and the number of false positives. A low sensitivity screening test may be compensated by more frequent screening. On the other hand, a test with low specificity will cause anxiety and unnecessary follow-up for people without a disease.

Further, prevalence is defined as the percentage of people in a population who have a condition such as a coronavirus infection. Positive and negative predictive values should be considered when evaluating the usefulness of a screening test or method. In the case of COVID-19 testing, a Positive Predictive Value (PPV) is the probability or percentage that at least one person in a room containing a group of people is an active spreader of the SARS-CoV-2 virus, and thus, the COVID-19 disease when a positive test result (e.g., from a NAAT assay) has been received.

An active spreader is a person who is actively transmitting the disease to others through viral particles in their exhaled breath. Scientific evidence suggests that most individuals that contract COVID-19 will actively spread the disease for 1-2 days prior to the onset of any disease symptoms. If, during this asymptomatic period, the person spends a significant amount of time in occupied spaces, perhaps involved in extended conversation, singing, exercising, or other activities that are known to result in higher-than-normal viral shedding, a “super spreader” event can occur. A super spreader event is said to have occurred when several, for example, five or more, people become infected from a single individual due to exposures that occur at the particular time period and location associated with the event.

In the case of COVID-19 testing, a Negative Predictive Value (NPV) is the probability that none of the subjects in the room are actively shedding SAR-CoV-2 viruses when a negative test result (e.g., from a PCR assay) is received. PPV and NPV are both dependent on sensitivity, specificity, and prevalence of COVID disease. If prevalence is high, for example, if at least 500 people per 100,000 are active spreaders, and typically 20 people are in the room being screened, then there is a 10% chance of a true positive result. If a diagnostic test has 99% sensitivity and 99% selectivity, and the test result (e.g., PCR) is positive, there is an 92% probability that the test is correct and at least one person in the room is truly shedding the virus. The primary assumption made in drawing this conclusion is that the viral particles in the collected sample came from the exhaled breath of the room's current occupants, and not from fomites or other potential sources of airborne viral particles. Fomites are objects or materials such as clothes, furniture, and utensils that are likely to carry pathogens such as viral particles. In the above examples, there is also an 8% possibility the test result is incorrect. As prevalence decreases, the positive predictive value decreases quickly. At about 3.3% prevalence for the first analysis system (e.g., a PCR assay with a 99% sensitivity and selectivity), a positive test result indicates that there is only a 77% chance that the sample is a true positive. On the other hand, if the test has only a 90% sensitivity and 90% selectivity, and the prevalence of true positives is 5%, then the test only has a 50% chance that a positive test result is truly positive.

The U.S. military has used a Dry Filter Unit, or DFU, for collecting bioaerosol samples comprising biothreat agents to prevent bioterrorism attacks. The DFU samples the air at approximately 1000 L/min, but the air is split between two 2-inch filters so that each filter collects at approximately 500 L/min. However, the DFU using in-line power (that is, it requires AC power), consumes about 1000 W and is not suitable for ACF use. Further, the DFU is heavy (>20 lb.) and large in size (about 1 cu. ft.) and is not portable. The DFU is also very loud when operated and requires sound mufflers to enable their use in populated spaces. The U.S. Department of Homeland Security employs the BioWatch program to detect bioterrorism threats. This program operates a network of air-monitoring collectors at multiple locations. BioWatch laboratories process and analyze filter samples to determine the presence of select biological agents. Air samples are collected over 24-hour periods and then analyzed using labor intensive laboratory-based protocols for sample extraction, sample preparation, and analysis. Data may be reported 2-3 days after sample collection. As a result, these methods and systems are not suitable for ACF of COVID-19 and other respiratory tract diseases in occupied indoor spaces.

There are currently no products or services in the market that enable rapid screening for respiratory diseases such as COVID-19 for a group of people in a short period of time (e.g., about one hour or less). A need exists for methods for rapid screening of a group of people in, and thus, on-site, such that personnel performing the test can determine if the group of people includes one or more spreaders of a respiratory disease. A need exists for high flow rate sample collection methods for collecting exhaled breath aerosols (EBA) from group of people, which can be coupled with diagnostic devices that support an assay that is fast, sensitive, specific and preferably, characterized by low cost per test. Such a system may be used for active case finding (ACF) of COVID-19 and other respiratory tract diseases. To be effective, a system for ACF should preferably be rapid and inexpensive on a “per person” basis. High flow rate air sampling within the indoor environment combined with a PON-NAAT device has the potential to be effective as an ACF tool. In some environments, sample pooling may be implemented to make the approach more cost-effective.

### BRIEF DISCLOSURE

Described herein are exemplary methods and systems to determine if one or more individuals in a group of people are actively spreading the SARS-CoV-2 virus or other respiratory pathogens in exhaled breath using high volume air sampling and a NAAT test.

Disclosed is an exemplary method for active case finding in an indoor space for a respiratory disease associated with a group of people who are present in the indoor space comprising collecting an aerosol sample comprising EBA from ambient air in the indoor space onto a filter substrate using a high flow rate hand portable aerosol sample collector system, extracting aerosol particles from the filter substrate on-site into a liquid sample, and analyzing the liquid sample using a NAAT analysis system on-site to confirm or eliminate the presence of the respiratory pathogen in the indoor space. The analyzing step may comprise analyzing the sample using technologies that comprise at least one of PCR, RT-PCR, isothermal nucleic acid amplification, and ELISA. The analyzing step may comprise analyzing the sample using isothermal nucleic acid amplification. The method may be characterized by a concentration factor (CF) of at least 350,000. The collecting step may further comprise moving the hand portable aerosol sample collector system proximate to the group of people during the collecting step. The method may further comprise the step of isolating and diagnosing each member of the group for the respiratory disease if the sample analysis confirms the presence of a respiratory pathogen by indicating a positive test result associated with the aerosol sample. The high flowrate aerosol sample collector system may be configured to move air at a flow rate of at least about 200 L/min through the filter substrate. The aerosol sample collection time using the aerosol sample collector system may be between about 10 min and 30 min. The time to obtain a diagnostic test result may be less than about 60 min. measured from the start of the aerosol sample collector system. The time to obtain a diagnostic test result may be less than about 30 min. measured from the start of the aerosol sample collector system. The on-site analysis system may be characterized by a positive predictive value of at least 50%. The exemplary method may further comprise the step of pooling multiple aerosol samples into one combined aqueous sample prior to analyzing using the on-site analysis system. The extracting step may comprise extracting aerosol particles from the filter substrate using an extraction fluid comprising between about 0.05% and 0.08 TWEEN 20 and between about 10 mM (molar) and 25 mM Tris in hydrochloric acid. The extracting step may comprise extracting aerosol particles from the filter substrate using an extraction fluid characterized by a pH of between about 7.5 and about 8. The extracting step may be completed in less than about 5 min. The extracting step may be completed in less than about 2 min. The extracting step may comprises removing the filter from the aerosol sample collector system, positioning the filter inside a tube having a predetermined volume of extraction fluid and capping the tube, and shaking the tube vigorously for 30 s. The volume of extraction fluid in the capped tube may be less than about 5 ml. The volume of extraction fluid in the capped tube may be less than about 1 ml. The volume of extraction fluid in the capped tube may be between about 4 ml and about 8 ml.

Disclosed is an exemplary hand portable aerosol sample collector system comprising a filter holder to support a filter substrate, a fan to pull ambient air comprising aerosol particles through the filter substrate at a flow rate selectable by an operator and for a sampling time selectable by an operator, and a housing to substantially enclose the filter holder and the fan wherein the system is configured to operate at a noise level of less than about 70 dB. The fan may be configured to pull air through the filter at a flow of between about 200 L/min and about 500 L/min. The system may be powered by a rechargeable battery pack. The filter substrate may comprise at least one of a polyester felt, electret filters, a fluoropolymer nanofiber nonwoven mat disposed on a cellulose acetate backing, and a combination thereof. The filter substrate may be coated with a water-soluble coating.

Disclosed is an exemplary sample extraction kit for extracting aerosol particles from a filter disposed in a high flow rate aerosol sample collector system comprising a pair of tweezers; and a capped tube having a predetermined volume of extraction fluid. The volume of the capped tube may be between about 25 ml and about 50 ml. The volume of the extraction fluid may be about 4 ml. The volume of the extraction fluid may be between about 1 ml and about 6 ml. The extraction fluid may comprise between about 0.05% and 0.08% TWEEN 20 and between about 10 mM (molar) and about 25 mM Tris in hydrochloric acid. The extraction fluid may comprise about 0.05% TWEEN 20 and about 10 mM (molar) Tris in hydrochloric acid. The extraction kit may be disposable. The extraction kit may have a unique bar code for identifying the kit.

Disclosed is an exemplary sample extraction kit for extracting aerosol particles from a filter substrate having aerosol particles comprising a capped centrifuge tube having a predetermined volume of extraction fluid and an insert component configured to receive the filter substrate and to be slidably disposed inside the centrifuge tube wherein the insert has a substantially open bottom end that allows the extraction fluid to enter the insert component.

Disclosed is an exemplary hand portable aerosol sample collector system comprising a filter holder to support at least one filter substrate, a fan to pull ambient air comprising aerosol particles through the filter substrate at a flow rate selectable by an operator and for a sampling time selectable by an operator, and a housing to substantially enclose the filter holder and the fan wherein the system is configured to operate at a noise level of less than about 70 dB. The system filter substrate may be between about 2 in. and about 3 in. in diameter. The filter substrate may comprise at least one of a polyester felt, electret filters, and a fluoropolymer nanofiber nonwoven mat disposed on a backing material, and a combination thereof. The backing material may comprise at least one of cellulose acetate, polypropylene, and nylon. The filter substrate may comprise polyvinyl acetate nanofiber disposed on a backing material. The backing material may comprise at least one of cellulose acetate, polypropylene, and nylon.

Other features and advantages of the present disclosure will be set forth, in part, in the descriptions which follow and the accompanying drawings, wherein the preferred aspects of the present disclosure are described and shown, and in part, will become apparent to those skilled in the art upon examination of the following detailed description taken in conjunction with the accompanying drawings or may be learned by practice of the present disclosure. The advantages of the present disclosure may be realized and attained by means of the instrumentalities and combinations particularly pointed out in the appendant claims.

### DRAWINGS

The foregoing aspects and many of the attendant advantages of this disclosure will become more readily appreciated as the same becomes better understood by reference to the following detailed description, when taken in conjunction with the accompanying drawings, wherein:

FIGS. 1A-C. (A) Perspective view, (B) cross sectional isometric view, and (C) perspective view showing filter access door open, respectively, of an exemplary high flow rate aerosol sample collector system.

FIGS. 2A-D. (A) Exploded view, (B) assembled view, (C) a first face of the filter holder that mates with the sample vial in the kit, and (D) a second face of the filter holder shows the filter housed in a recess of the filter holder, respectively, of an exemplary aerosol particle extraction kit.

FIG. 3. Perspective and cross-sectional views showing filter access door open of another exemplary high flow rate aerosol sample collector system.

FIG. 4. Schematic diagram of an exemplary ACF method using a high flow rate aerosol sample collector system and a NAAT device.

FIG. 5. Perspective views of an exemplary aerosol particle sample extraction kit suitable for extraction using an exemplary extraction method using centrifugation.

FIGS. 6A-B. (A) Particle extraction efficiency measurements using exemplary fluid extraction methods, and (B) Box and whisker plots of normalized concentration measurements using three extraction methods for eluting captured aerosolized Bacteriophage MS on a filter using an exemplary sample collector system.

FIGS. 7A-C. (A) Concentration factor (CF) as a function of sample time of virus particles in indoor ambient air using an exemplary high flow rate aerosol sample collector system, (B) CF measured during capture of Bg spores using different filter materials and (C) CF measured during capture of Bg spores using an exemplary dissolvable nanofiber filter.

FIG. 8. Comparative viral titer results of aerosolized bovine coronavirus (BCoV) collected and extracted using exemplary aerosol sample collector system and other systems.

All reference numerals, designators and callouts in the figures are hereby incorporated by this reference as if fully set forth herein. The failure to number an element in a figure is not intended to waive any rights. Unnumbered references may also be identified by alpha characters in the figures.

The following detailed description includes references to the accompanying drawings, which form a part of the detailed description. The drawings show, by way of illustration, specific embodiments in which the disclosed systems and methods may be practiced. These embodiments, which are to be understood as “examples” or “options,” are described in enough detail to enable those skilled in the art to practice the present invention. The embodiments may be combined, other embodiments may be utilized, or structural or logical changes may be made, without departing from the scope of the invention. The following detailed description is, therefore, not to be taken in a limiting sense and the scope of the invention is defined by the appended claims and their legal equivalents.

In this disclosure, “aerosol” generally means a suspension of particles dispersed in air or gas. “On-site” generally means proximate to the space being sampled for a respiratory disease. A “proximate space” is one that is within a about 5 min. travel time to transport a sample from the indoor space where the sample was collected to the location where the sample will be analyzed. “Proximate” within an indoor space means at a distance of about 6 ft. or less from a person. “Rapid” generally means in about one hour or less. “Indoor space” generally means any enclosed area or portion thereof. The opening of windows or doors, or the temporary removal of wall panels, does not convert an indoor space into an outdoor space.

The terms “a” or “an” are used to include one or more than one, and the term “or” is used to refer to a nonexclusive “or” unless otherwise indicated. In addition, it is to be understood that the phraseology or terminology employed herein, and not otherwise defined, is for the purpose of description only and not of limitation. Unless otherwise specified in this disclosure, for construing the scope of the term “about,” the error bounds associated with the values (dimensions, operating conditions etc.) disclosed is ±20% of the values indicated in this disclosure. The error bounds associated with the values disclosed as percentages is ±5% of the percentages indicated. The word “substantially” used before a specific word includes the meanings “considerable in extent to that which is specified,” and “largely but not wholly that which is specified.”

### DETAILED DISCLOSURE

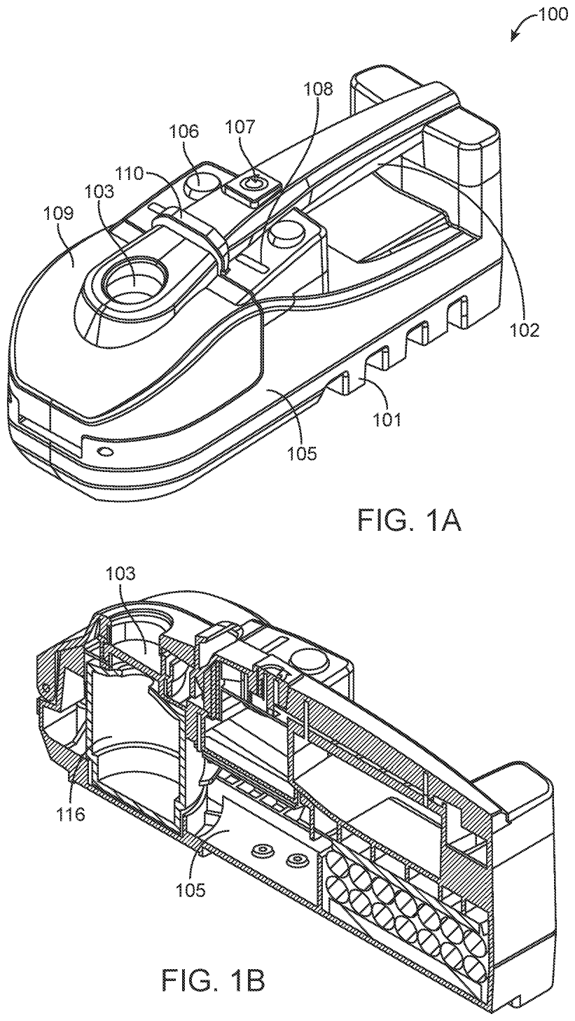

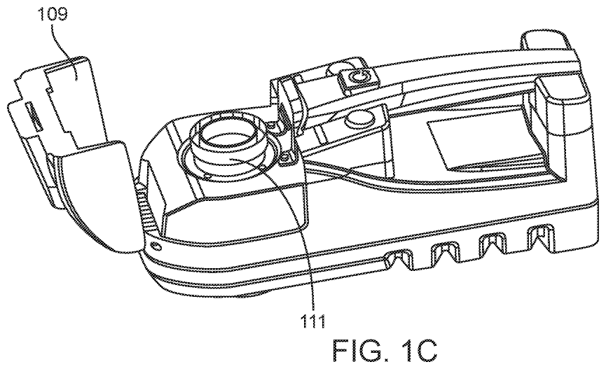

The exemplary methods and systems disclosed herein may be used for active case finding of active spreaders of a respiratory disease such as COVID-19. FIGS. 1A-C show an exemplary sample capture (or collector) system 100 for use in an exemplary ACF diagnostic method that does not use an impactor but instead uses a filter cartridge-based collector. System 100 comprises housing 101 that substantially encloses the components required for capturing or collecting aerosol particles. Housing 101 may include an integral handle 102. Ambient air containing aerosol particles is drawn into system 100 through fluid inlet 103, passes through filter 104 provided to trap aerosol particles, and exits out of system 100. An air outlet is also provided (not shown) that enables air to exit system 100 after most of the particles are trapped by the filter. Exemplary filter 104 may comprise polyester felt materials that are capable of capturing particles with particle size of at least about 1 μm. Another exemplary filter are electret filters which are known to capture virus particles. The electret filters are capable of removing particulate matter by strong electrostatic forces generated by the electret fibers that make up these filters. The embedded charge on electret filters is believed to make the filter surfaces to attract viruses, which generally also carry surface charges. Alternately, filter 104 may comprise a plurality of different types of filters assembled in a sandwich-type assembly that is capable of capturing a wide range of particle sizes and particle types from ambient air drawn into system 100. Filter 104 may comprise a fluoropolymer nanofiber nonwoven mat disposed on a backing material comprising at least one of cellulose acetate, nylon and polypropylene. Exemplary nanofiber filters may be characterized by average pore diameter of about 3.97 microns, bubble point pore diameter of about 4.95 microns and bubble point pressure (pressure at largest pore) of about 2.36 psi. The pore diameter at maximum pore size distribution may be about 2.44 microns. Exemplary filters may also comprise coatings that are applied to the filters. Examples of coatings are hydrophilic or water-soluble coatings that make the particles to be removably attached to the fibers of the filters. Alternately, these coatings may enable the particles to be easily extracted from the filters using suitable extraction methods described below. Exemplary coatings suitable for collection of aerosols and which enable particle extraction are disclosed in U.S. Pat. No. 6,363,800 and titled “COATING TO ENHANCE THE EFFICIENCY OF A PARTICLE IMPACT COLLECTOR,” which is incorporated by reference herein in its entirety.

System 100 is preferably powered by a rechargeable battery pack housed in battery compartment 105. An exemplary Li-ion battery has a rated capacity of about 3.5 Ah at 25.4V (nominal). The charging voltage is about 29.4 V. The maximum continuous discharge rate is about 6A, corresponding to about 150 W. The compact and lightweight battery weighs about 760 g and measures about 135 mm×68.5 mm×46.5 mm. Air is drawn through inlet 103 using a suitable low-power, and low-noise fan or blower (not shown) that is disposed downstream of filter 104 in fan compartment 116. An exemplary fan is capable of moving air through the filter at flow rates of between about 200 L/min (liters per minute) and about 500 L/min. A centrifugal fan may be used in system 100. The blower (or fan) is powered by the battery and may comprise a 24 VDC high-speed, high-pressure vacuum double-layer fan. Maximum current draw may be about 7A, resulting in a fan speed of about 22,000 rpm in an open flow configuration. When installed in exemplary system 100, the maximum fan speed is typically about 25,800 rpm at 3.4 A and generating 10.5 kPa of pressure. An exemplary fan is characterized by a lifetime of at least about 10,000 h. Sampling times and flow rates may be selected by the user or operator prior to starting the system using toggle switch 106. Sampling times may be varied between 5 min. and 30 min. in increments of 5 min. System 100 also comprises a system start/stop button 107. System 100 may also comprise battery charge level indicator 108 that alerts the user or operator to recharge the batteries or insert another charged battery pack. Any type of battery pack may be employed including Li-ion and lead acid rechargeable batteries. System 100 may comprise filter access door 109 which may be released from housing 101 using latch 110. Inlet 103 is in fluid communication with filter holder 111 which house a filter substrate 104. Filter holder 111 may be removably disposed in system 110. Filter holder 111 may be made of any hard material suitable for compressing and holding the filter in place. Plastic or metal may be used to fabricate the holder, but plastic is preferred because it generally has a higher strength-to-weight ratio, and decreasing weight is a key requirement. After sampling, holder 110 with filter 104 may be transferred to a sample extraction kit for extracting the trapped aerosol particles from filter 104. Filter 104 may have a nominal diameter of about 2 in. Exemplary system 100 may be characterized by a particle capture (collection) efficiency of between about 75% and about 95%.

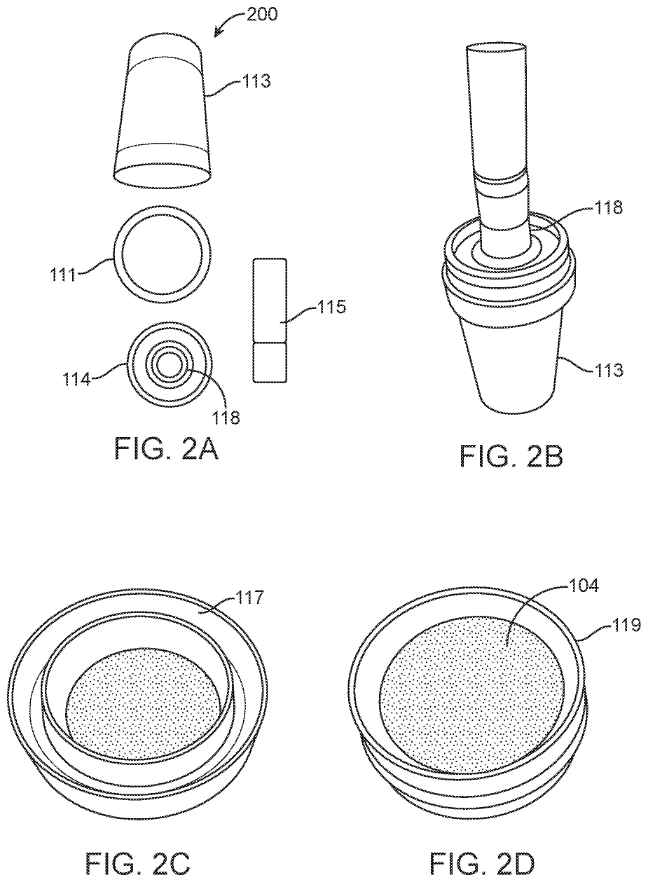

The fan in collector system 100 may be capable of moving up to about 500 L/min for 5 min. for quick sampling when operated in a burst-mode, to quickly sample air in indoor spaces. Collector system 100 is capable of collecting a sample over various sampling times that may be set by the user or operator using sample time selector 106. As previously described, nominal sampling time selections are user-selected and may include the options of selecting sampling times of 5 min., 10 min., 15 min., 20 min., 25 min., and 30 min. the standard time is 5 min. A typical sampling time may be 5 min, because in the event of a suspected pandemic causing virus, it is imperative to quickly collect a sample that is representative of the indoor space and complete a sample analysis with high specificity and sensitivity to isolate a spreader and limit the spread of the disease. A high flow rate air sampler provides a sample that is more representative of the entire space being sampled. After sampling, captured aerosol particles may be extracted using a suitable sample extraction kit 200 (FIGS. 2A-D). An exemplary sample extraction kit 200 may comprise a vial 113, adapter component 114, and an extraction fluid cartridge 115. Filter holder 111 may be provided with groove 117 on one side to snap fit or press fit with the opening of vial 113. On the opposite side 119, filter holder 111 is configured to support filter 104 in a recess and is also configured to engage with extraction kit adapter component 114. Adapter component 114 has a first flat face that is configured to interface with filter 104 and evenly distribute the extraction fluid across the surface of filter 104. On the face opposite to the first flat face, adapter component 114 has a boss type opening 118 that is configured to receive cartridge 115 and to fluidly connect cartridge 115 to vial 113 through filter 104.

In an exemplary sample extraction method using extraction kit 200, filter holder 111 with filter 104 is removed from sample capture system 100 and the captured aerosol sample is extracted into vial 113 to provide a liquid sample containing the captured aerosol particles for subsequent analysis. Filter holder 111 with the aerosol sample may be removably snap-fit on to receiving vial 113. Adapter component 114 is then press-fit to the filter holder and cartridge 115 is fit into opening 118 in adapter 114. The extraction fluid in cartridge 115 may comprise about 0.075% TWEEN 20 and about 25 mM (molar) of Tris. Extraction cartridge 115 may hold between about 4 ml and about 8 ml of extraction solvent or fluid. TWEEN 20 is a polysorbate 20 surfactant. Tris is tris(hydroxymethyl)aminomethane and is commonly used as a component in buffer solutions. The extraction fluid is then forced out from the cartridge by manually applying pressure to the cartridge, for example, by pressing down on the bottom end of the cartridge. Extraction fluid flows out of cartridge 115, spreads across the surface of filter 104 and elutes captured aerosols from the filter into vial 113 to provide between about 3 ml and about 5 ml.

In exemplary system 100, an impactor collector and associated rinsing mechanisms or components to enable rinsing of the impactor between successive samples are not needed because the aerosol particles are captured directly on the filter. System 100 is essentially free from cross contamination issues. Cross-contamination is more problematic when the concentrated hydrosol (aerosol collected and concentrated into a small volume of liquid) of the current sample comes in contact with a surface that has contacted previous hydrosol samples. System 100 and the exemplary sample extraction method described above is essentially free from cross contamination issues.

The volume of extraction fluid and the overall collection efficiency into the liquid sample is important to the overall sensitivity of the measurement. Further, a concentration factor (CF) for a sample collector system may be defined by the following formula:

CF=E*Fₐᵢᵣ*t/Vₛₐₘₚₗₑ

where E is the efficiency of the sampling process for collecting aerosol particles in air into a liquid, Fₐᵢᵣ is the flowrate of air being sampled, t is the sampling time period, and Vₛₐₘₚₗₑ is the final volume of the liquid sample collected. CF may be considered to be a figure of merit for an aerosol sample collector and extraction system and may be viewed as the ratio of concentration of particles in the extraction fluid to the concentration of particles in air. To increase CF, the volume of the extraction fluid should be minimized. Further increasing the air flow rate and increasing sampling time would also increase CF. An extraction fluid volume of between about 4 ml and about 8 ml is preferred. A concentration factor of at least 200,000 is targeted for the exemplary method to achieve the desired limit of detection. A concentration factor of between about 350,000 and 500,000 is preferred. Efficiency E is a product of the sample collector system efficiency and efficiency of extracting particles from the filter into a liquid and is at least about 80% and may be increased to between about 85% and about 90% by optimizing the properties of the filter material. Since efficiency E is dependent on the filter material, concentration factor CF is a function of the air flow rate and sampling time beside extraction fluid volume. As previously discussed, short sampling times of about 10 min. and preferably, about 5 min. and increased flow rates (at least about 200 L/min) are preferred.

As disclosed above, increasing flow rate through exemplary sample collector system 100 and sampling period (collection time) would increase the concentration factor. However, the need to increase air flow rate should be balanced with other factors such as the size of the fan (or blower) in the exemplary sample collector systems, the noise levels associated with operating a larger fan, the increased power requirement for running a larger fan and required increase in battery capacity, which may increase the weight of the system. Since exemplary aerosol sample collector system disclosed herein are preferably hand-held or portable devices powered by a battery, consideration of the above factors may not support continuous air flow rate of, for example, about 1000 L/min. Operating the exemplary collector systems in a burst mode for a short period, air flow rate of about 1000 L/min may be feasible, and the flow rate may be reduced to about 400 L/min or 200 L/min thereafter. As is well known, battery technology is continuously improving as batteries with increasing specific energy (W-h/kg) are periodically being launched on a commercial scale. With the availability of light-weight and high specific energy batteries that can support increased power draw from the fan, air flow rates that exceed 400 L/min, for example flow rates of between about 400 L/min and about 1000 L/min, and flow rates of least about 1000 L/min, is within the scope of the exemplary sample collector systems disclosed herein. Increasing flow rates may also require an increase in the diameter of filter 304 to decrease pressure drop through the filter. For example, the diameter of filter 304 may be increased from about 2 in. to about 3 in., which in turn may require increasing the volume of extraction fluid.

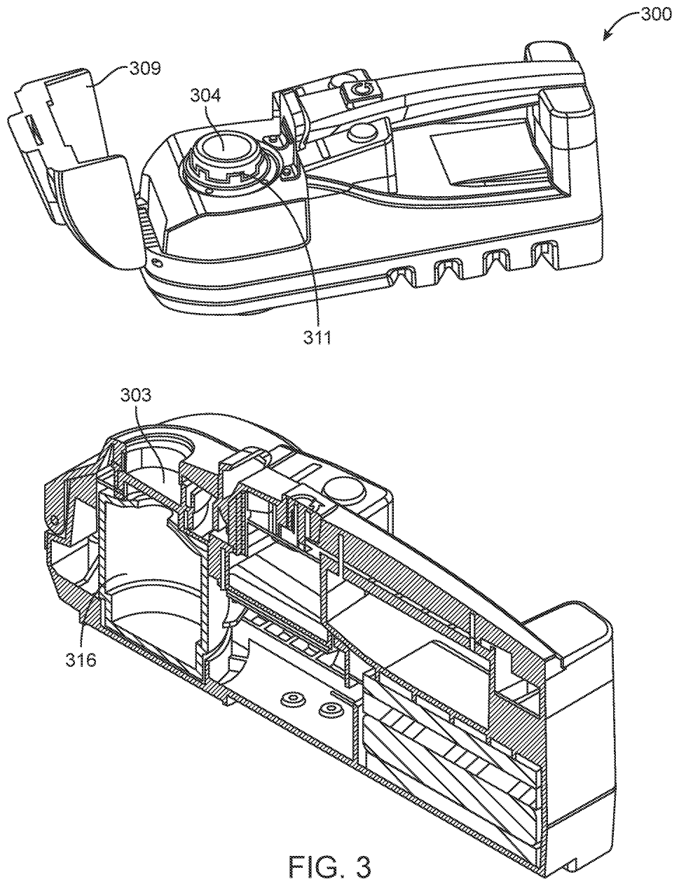

In another exemplary aspect of system 100, sample capture (collector) system 300 (FIG. 3), for use in an exemplary ACF diagnostic method, may be configured to move air containing aerosol particles through inlet 303 into the system at a high flow rate of at least about 200 L/min during prolonged sampling times of up to about 30 min. In contrast to system 100, filter holder 311 in system 300 is configured to support filter 304 having a surface area of about 25 cm² such that filter 304 may be easily removed, for example, using a pair of tweezers after opening filter access door 309. Filter holder 311 need not be removable from system 300. An exemplary air flow rate may be between about 200 L/min and 500 L/min. In exemplary system 300, the air mover (fan) housed in compartment 316 may consume only about 50 W to about 100 W of power. This high flow rate of air permits air sampling in areas where the density of people is high or where in places where people tend to congregate. High density areas may include security lines at airports, break rooms, cafeteria, auditoria, conference rooms, sports stadiums, airport boarding gates and the like. A high flow rate of at least about 200 L/min is also advantageous for indoor air sampling in large rooms because the sample step can be completed quickly. A large room, with approximate dimensions of 10 m×10 m×3 m might contain approximately about 250 cubic meters of air. In about 5 min., a 500 L/min aerosol collector system may extract aerosol particles (e.g., viruses) from approximately 1% of the air in the room, which represents a reasonable sample since most rooms are reasonably well mixed on account of the movement of people and the movement of air by the ventilation (HVAC) system. A low flow rate sampler, for example, operating at 100 L/min, would take almost 25 min. to sample the same volume of air. Since reducing sampling time is critical, keeping sampling times to less than 10 min., and preferably, to about 5 min. is beneficial for many PON-ACF applications. Assuming a 5 min. sampling time, a high flow rate portable sample collector system capable of drawing about 400 L/min as the operator moves around the room proximate to people present, leads to a more representative sample and mitigates any issues with non-uniform distribution of aerosols in the room.

Exemplary system 300 may be configured to operate at noise levels that approximate ambient noise. Noise levels at 500 L/min of air flow may be about 70 dB, and preferably, below about 60 dB at lower flow rates that enables sample collection without disrupting normal communication or distracting the room's occupants, and potentially creating concern or panic. System 300 is powered by a rechargeable battery pack, is hand portable and can operated for about 4 h on a full charge. Battery recharging may be done while the battery pack is housed inside the system. System 300 with the battery, may weigh between about 5 lb. and 10 lb. Light-weighting of system 300 may be done by optimizing the selection and properties of filter 304, pressure drop through the filter at high flow rates, of the low-power fan, and a battery with a high specific energy (W-h/kg). Filter 304 may comprise a fluoropolymer nanofiber nonwoven mat disposed on a suitable backing material. The backing material may comprise at least one of cellulose acetate, nylon, and polypropylene. Exemplary nanofiber filters may be characterized by average pore diameter of about 3.97 microns, bubble point pore diameter of about 4.95 microns and bubble point pressure (pressure at largest pore) of about 2.36 psi. The pore diameter at maximum pore size distribution may be about 2.44 microns. Further, system 300 may be between about 1 cu. ft. and 0.25 cu. ft. in size.

In another exemplary aerosol particle extraction method, the filter from exemplary sample collector system 100 or 300 may be removed and positioned inside a syringe (e.g., 25 ml to 30 ml syringe) using a pair of tweezers or other means. Between about 4 ml and about 8 ml of extracting fluid from a small tube or container (e.g., a 50 ml capped tube) may be pulled into the syringe through the filter by moving the plunger of the syringe. The extraction fluid may comprise about 0.05% TWEEN 20 and about 10 mM (molar) of Tris in hydrochloric acid. The pH of the extraction fluid may be between about 7.5 and about 8. The pH of the extraction fluid may be about 7.8. The filter in then soaked in the extraction fluid for between about 2 min. and 5 min. During the soaking period, the syringe may be inverted up-and-down a few times. The soaking period may be about 4 min. The fluid with the aerosol particles is then pushed out of the syringe by moving the syringe plunger down, and into a vial or a capped tube, for example, a 50 ml capped tube. Alternately, filter 304 with captured aerosol particles may be placed in suitable extraction fluid in a centrifuge tube and extracted using a centrifuge. Alternately, the filter with captured aerosol particles may be placed in an extraction fluid in a suitable tube along with quartz beads and manually shaken (or placed in an ultrasonic bath) or centrifuged to extract the particles into the fluid. An exemplary sample extraction kit may comprise a syringe, a pair of tweezers, and a capped tube comprising extraction fluid. The volume of the syringe may be between about 25 ml and 30 ml. The volume of the capped tube may be about 50 ml. The volume of the extraction fluid may be between about 4 ml and 8 ml.

Disclosed is another exemplary aerosol particle extraction method comprising removing the filter 104 or 304 from the aerosol sample collector system, inserting the filter into a vial or tube having a small volume of extraction fluid and manually shaking the tube vigorously for about 30 s after capping the tube. The volume of the capped tube may be between about 25 ml and about 50 ml. The volume of the capped tube may be about 25 ml. The extraction fluid may comprise about 0.05% TWEEN 20 and about 10 mM (molar) of Tris in hydrochloric acid. The extraction fluid may comprise between about 0.05% and 0.08% TWEEN 20 and about 10 mM (molar) Tris in hydrochloric acid. The pH of the extraction fluid may be between about 7.5 and about 8. The pH of the extraction fluid may be about 7.8. The volume of the extraction fluid may be between about 1 ml and about 10 ml. The volume of extraction fluid in the capped tube may be between about 1 ml and about 6 ml. The volume of extraction fluid may be about 4 ml. The volume of extraction fluid may be less than about 5 ml. The volume of extraction fluid may be less than about 1 ml. The vial or tube may be a conventional capped centrifuge tube.

Disclosed is an exemplary sample extraction kit 200 for use in a diagnostic method for active case finding of respiratory disease comprising a syringe, a pair of tweezers, and a capped tube comprising extraction fluid. The volume of the syringe may be between about 25 ml and 30 ml. The volume of the capped tube may be about 50 ml. The volume of the extraction fluid may be between about 4 ml and 8 ml. The extraction fluid (sterile buffer solution) may comprise between about 0.05% and 0.08% TWEEN 20 and between about 10 mM (molar) Tris in hydrochloric acid. The extraction fluid may comprise about 0.05% TWEEN 20 and about 10 mM (molar) Tris in hydrochloric acid. The extraction kit may be disposable. The extraction kit or sample vial may have a unique bar code or RFID tag for sample tracking.

Disclosed in another exemplary sample extraction kit for extracting aerosol particles from a filter disposed in a high flow rate aerosol sample collector system comprising a pair of tweezers, and a capped tube having a predetermined volume of extraction fluid. The volume of the capped tube may be between about 25 ml and about 50 ml. The volume of the capped tube may be about 25 ml. The extraction fluid may comprise about 0.05% TWEEN 20 and about 10 mM (molar) of Tris in hydrochloric acid. The extraction fluid may comprise between about 0.05% and 0.08% TWEEN 20 and about 10 mM (molar) Tris in hydrochloric acid. The pH of the extraction fluid may be between about 7.5 and about 8. The pH of the extraction fluid may be about 7.8. The volume of the extraction fluid may be between about 1 ml and about 10 ml. The volume of extraction fluid in the capped tube may be between about 1 ml and about 6 ml. The volume of extraction fluid may be about 4 ml. The vial or tube may be a conventional capped centrifuge tube. The extraction kit may be disposable. The extraction kit or sample vial may have a unique bar code or RFID tag to enable sample tracking purposes.

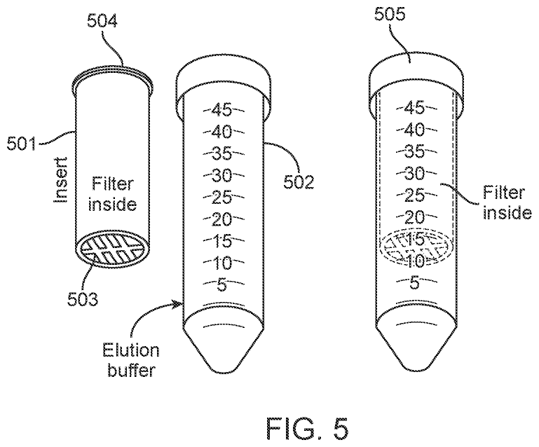

In another exemplary aerosol particle extraction method using a centrifuge, the filter from exemplary sample collector system 100 or 300 may be removed and positioned inside insert 501 (FIG. 5). Insert component 501, which may be made of plastic, has a grated or meshed bottom end 503 and is configured to slide inside centrifuge tube 502 having a volume of about 50 ml. Top end 504 is disposed opposite to bottom end 503 of insert 501 and has a rim or lip that prevents the filter from falling out of the insert. Capped tube 502 may contain about 8 ml of sterile buffer solution (aerosol extraction fluid) comprising about 0.05% TWEEN 20 and about 10 mM Tris and may be characterized by a pH of between about 7.5 and about 8.0. The pH of the extraction fluid may be about 7.8. Bottom end support 503 supports the filter inside the insert but also allows extraction fluid from entering inset 501 through the meshed or grated end and contacting the filter. Any similar insert component shaped in the form of a basket that supports the filter and allows for the fluid to enter the insert and contact the filter may be used. After receiving the insert having the filter with trapped aerosol particles, capped tube 502 is closed with cap 505. The filter may be soaked with the extraction fluid by inverting the tube back-and-forth a few times. Capped tube 502 is then placed in a low-speed centrifuge and then run at about 3000 rpm for about 5 min. Capped tube is removed from the centrifuge and the insert with the filter inside is removed from capped tube 502. The liquid sample in the tube may then analyzed using a portable PCR analysis system or other suitable analysis techniques if the sample is analyzed in a laboratory. Several portable centrifuge systems are commercially available and may be adapted to receive 50 ml centrifuge tubes and run on-site (e.g., Sprout centrifuges sold by Heathrow Scientific).

The exemplary extraction methods disclosed may also use filter materials that are dissolvable into the extraction fluid. An exemplary filter material that may dissolved in the extraction fluid is polyvinyl acetate (PVA). A PVA filter of about 2 in. nominal diameter may be dissolved in between about 0.5 ml and about 1 ml of extraction fluid. A PVA filter of about 3 in. diameter may be dissolved in between about 1 ml and 2 ml of extraction fluid. An exemplary dissolvable filter is polyvinyl acetate nanofibers disposed on a backing material. The backing material may comprise at least one of cellulose acetate, nylon, and polypropylene. The extraction fluid may also comprise additives to inactivate pathogens such as viruses and bacteria and to stabilize the nucleic acids (e.g., RNA) of these pathogens.

Other types of aerosol sample collectors that are capable of moderate or high flow rates may also be used if weight, size and power consumption are not an issue. The SpinCon® II wetted-wall cyclone (InnovaPrep Inc., MO), which operates at up to 500 L/min may be used. However, this system is quite large (>1 cu. ft.), and heavy (>50 lb.), and requires 800 W of power. The Coriolis Micro wetted-wall cyclone (Bertin, France), which operates at a flow rate of up to 300 L/min may also be used. These collectors provide approximately 10 ml of aqueous sample. Further, virtual impactors may be combined with an impinger or wetted surface impactors. The XMX collector (Dycor, Inc., Canada) incorporates this approach and operates at approximately 530 L/min. However, this system is large (>1 cu. ft.) and heavy (60 pounds), and required about 250 W. It is also noisy and generates about 100 dB of sound at 1 meter. In general, these sample collectors are large, heavy, noisy, and are prone to cross contamination and require a cleaning step between successive samples. Other sample collectors (e.g., the Bertin Coriolis Micro system) require greater than about 100 W of power but can sample only at lower flow rates (e.g., 300 L/min or less).

An exemplary diagnostic system may comprise one of the exemplary high flow aerosol sample capture systems described herein and a NAAT sample analysis system. Exemplary sample analysis systems include the GeneXpert (Cepheid, Inc., Sunnyvale, Calif.) and FilmArray (BioFire Diagnostics, Inc., Salt Lake City, Utah), which may be used to provide a diagnostic result in about 45 min. Both devices are exemplary genomics-based point of need (PON) diagnostic assay instruments and use PCR technology. The exemplary diagnostic system performs ACF of COVID-19 and other respiratory diseases on-site, or at the “point of need” (PON). PCR-based diagnostic tools enable rapid, low-cost point-of-need assays for several diseases including respiratory tract diseases such as COVID-19. Another NAAT device with similar sensitivity to a PCR device is Abbott ID NOW (Abbott Laboratories, Abbott Park, Ill.) which uses isothermal nucleic acid amplification. The Abbott ID NOW device has shown ≥94.7% sensitivity (positive agreement) and ≥98.6% specificity (negative agreement) when compared to two different lab-based PCR reference tests for detection of nucleic acid from the SARS-CoV-2 virus. The ID NOW device is portable and allows for fast diagnosis of COVID-19 samples with and outputs results in less than about 15 min. Using the ID NOW device for analyzing aerosols extracted into an extraction fluid using the exemplary sample collector system and extraction methods disclosed herein may provide a diagnostic test result in less than about 60 min. measured from the starting of the aerosol sample collector system. The time to obtain a diagnostic result measured from the starting of the aerosol sample collector system may be less than about 30 min. For example, an assay using the Abbott ID NOW may be completed in under 15 minutes. When combined with a 10 min. sample collection time using the exemplary aerosol sample collector systems disclosed herein, and an extraction time of less than about 2 min. the entire ACF test may be completed in less than about 30 min.

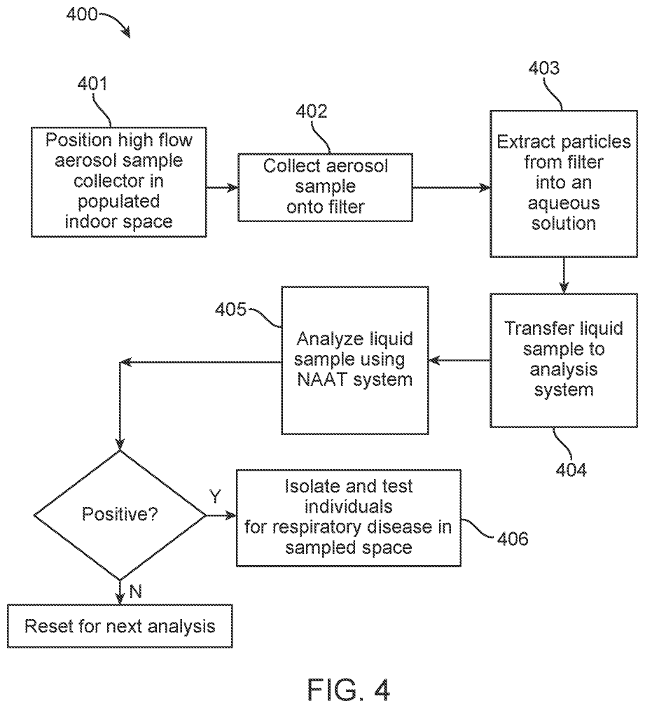

In an exemplary ACF method 400 (FIG. 4), a high flow rate sample collector system, for example, exemplary system 300 may be placed in an indoor space occupied by a group of people in step 401. In step 402, the sample collector system is run for a predetermined sampling time to collect aerosol particles on a sample filter housed inside the sample collector system. The sample is collected proximate to the people present in the room. The sampling time may be between about 5 min and about 30 min. As previously discussed, short sampling times of about 10 min. and preferably, about 5 min. and high air flow rates are preferred. The flow rate through the sample collector system may be between about 200 L/min and about 500 L/min. In step 403, the aerosol particles may be extracted using an extraction fluid using one of the exemplary methods described previously. The liquid sample may then be transferred to a sample analysis system in step 404. A nucleic acid amplification technique (NAAT) such as a PCR analysis system is preferred (e.g., FilmArray®) or ID NOW because it is field operable and allows for sampling and quantification of virus particles such as the SARS-CoV-2 virus in less than about 1 h with high specificity and sensitivity when used in conjunction with a high flow aerosol sample collector system. The sample analysis is then run to output a test result in step 405. If the test result is positive for suggesting the presence of aerosol pathogen such as the SARS-CoV-2 virus, the people in the indoor space are isolated for further individual testing as needed in step 406. The aerosol collector system would not typically require decontamination between successive aerosol particle collection steps. A disinfecting wipe may be used to wipe down the exterior surfaces of the aerosol collector. The disclosed exemplary on-site analysis method may be characterized by a positive predictive value of at least 50%, and more preferably, of at least 90%.

The liquid sample obtained using an exemplary high flow rate sample collector system may be collected from one system which may be carried around the room to collect a spatially representative sample. Alternately, aerosol samples may be collected at multiple fixed points using a plurality of collector systems and the liquid samples extracted from each system may be pooled (combined) and then analyzed. In a building (indoor space), the exemplary sample collector systems may be placed near HVAC (air) ducts to sample air. In exemplary system 300, the liquid sample may be collected in a “consumable” package and then analyzed using a suitable analysis system such as a PCR. The exemplary systems may be configured to be remotely operated (started and stopped) at predetermined times by replacing the start/stop button 107, for example, with a suitable input enable signal, for example, a 12V signal.

### EXAMPLES

### Example 1: Scenario Analysis for Indoor Air Sampling: Key Metrics Related to COVID-19 Related Virus Sampling and Analysis

Table 1 summarizes sensitivity and specificity of an exemplary ACF diagnostic system comprising exemplary aerosol sample collector system 300 and a FilmArray™ PCR device. Sensitivity and specificity are characteristics of the test and are independent of the population being testing. The sensitivity of the respiratory panel for the FilmArray™ PCR system has been tested and shows very high sensitivity and specificity (Table 1) for clinical samples. It is anticipated to have similarly high values for indoor aerosol samples.

In the exemplary systems and methods disclosed, sensitivity measures whether an air sample can correctly identify if a population that includes one or more persons is actively shedding a virus that causes a respiratory disease such as COVID-19. Specificity relates to measuring whether the PCR test on an air sample can correctly identify a sample that is negative for the presence of one or more active spreaders of the disease. A True Positive is a test result on a sample from a group of people with an active spreader. A True Negative is a negative test result related to an air sample from a group of people without an active spreader. A False Positive is a positive test result on an air sample from a group of people without an active spreader in the room and a False Negative is a negative test result on an air sample from a group of people without an active spreader. The results shows that PCR has high sensitivity and specificity for bioaerosol analysis.

TABLE 1
Exemplary Scenario Analysis - Summary

| Prevalence | True +ve | False +ve | False −ve | True −ve | PPV | NPV | Sensitivity | Specificity |
|---|---|---|---|---|---|---|---|---|
| 2% | 96 | 96 | 2 | 4800 | 50% | 100.0% | 98% | 98% |
| 11% | 96 | 16 | 2 | 800 | 86% | 99.8% | 98% | 98% |
| 49% | 96 | 2 | 2 | 100 | 98% | 98.0% | 98% | 98% |

### Example 2: Detection Limits for Virus Particle Sampling and Analysis Using Exemplary System 300 and a NAAT Device

A number of recent publications have found that airborne virus loadings in areas known to have COVID-positive patents are often in the 1-10 copies per liter of air range. Assuming the SARS-CoV-2 virus is present in the ambient air at a concentration of 1 copy per liter of air, a 5 min. sample at 500 L/min will capture at most 2500 copies of the virus. Assuming 2000 copies of the virus are collected and extracted from the filter in exemplary collector system 100 or 300 according to the exemplary method described herein, the overall particle collection efficiency is about 80%. The sample may then be extracted into 5 ml of aqueous solution, which would yield a concentration of virus in the sample of 400 copies/ml. FilmArray PCR device requires 300 μl per assay and has a lower detection limit of about 330 copies/ml. Therefore, the exemplary sample capture system operating at about 500 L/min yielding about 80% overall collection efficiency of SARS-CoV-2 virus should easily support a ACF diagnostic system that includes a PCR analysis system.

Alternately, the concentration of virus particle (e.g., SARS-CoV-2) in indoor ambient air may be between about 1 copy/liter and 1000 copies/liter of indoor ambient air. Exemplary aerosol sample capture system 300 may be used to collect ambient air having 1 copy/liter of virus particles at a flow rate of about 200 L/min through the system over a collection time of 5 min. If 80% of virus particles captured using filter 304 are extracted into about 4 ml extraction liquid, the concentration of the virus particles in the liquid is 200 copies/mL, which is well within the 160-300 copies/mL range of NATT devices such as the FilmArray™ device and within the 125 copies/mL detection limit for the Abbot ID NOW device. Aerosol sample collection using exemplary system 300 and sample analysis using a FilmArray® or ID NOW NAAT device may be used to detect as a low as 1 copy/liter of virus particles such as the SARS-CoV-2 virus in air. At 10 min. sample collection time using exemplary device 300, the concentration of virus particles in the liquid may be about 400 copies/mL. The extraction fluid may a water-based viral inactivator and may comprise between about 0.05% and about 0.08% TWEEN 20 and between about 10 mM (molar) and about 25 mM Tris in hydrochloric acid. Alternately, an extraction fluid that keep the captured virus particles and other microbes in a viable and stable state may be used for subsequent culture in a suitable culture medium. The filter 304 in device 300 may comprise at least one of polyester felt, electret filters, a fluoropolymer nanofiber nonwoven mat on a backing material, and a combination thereof. The backing material may comprise at least one of cellulose acetate, nylon, and polypropylene. Exemplary nanofiber filters may be characterized by average pore diameter of about 3.97 microns, bubble point pore diameter of about 4.95 microns and bubble point pressure (pressure at largest pore) of about 2.36 psi. The pore diameter at maximum pore size distribution may be about 2.44 microns.

### Example 3: Particle Extraction Efficiency and Extraction Efficiency of MS2 Phage

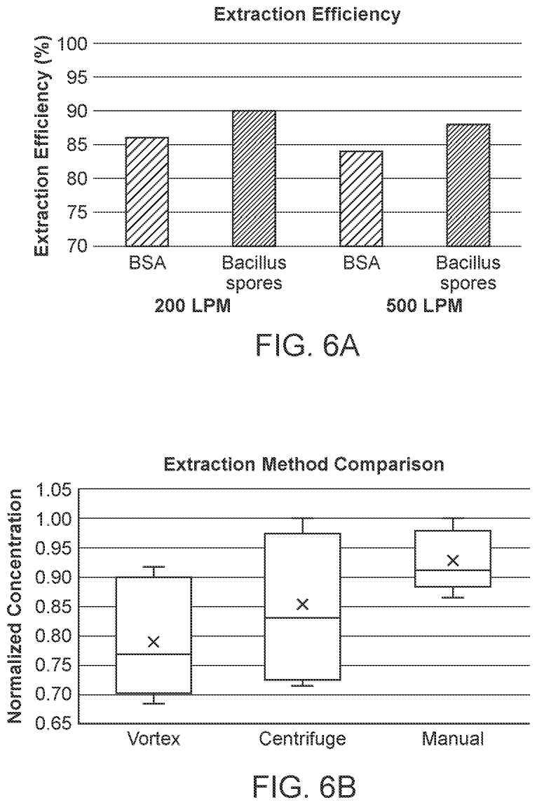

FIG. 6A shows the results of extraction efficiency measurements using the exemplary extraction methods demonstrating extraction efficiency of about 85% for both Bacillus spores and bovine serum albumin (BSA) protein at two different flow rates. Assuming the filter capture efficiency using exemplary system 300 is about 95% for these particles with size varying between about 1 μm to about 5 μm, an overall collection efficiency of 80% may be realized even at high flow rate sample collection using flow rates of about 400 L/min.

Extraction of Bacteriophage MS2 captured using exemplary system 300 was examined using three extraction methods, namely, employing a vortex mixer/shaker, a centrifuge, and manual shaking of a vial containing the buffer solution and filter. A solution containing MS2 phage was aerosolized using a 6-jet collision nebulizer. MS2 phage was selected as a surrogate for SARS-CoV-2 because it is a non-pathogenic RNA virus. The sample aerosol was injected into an 8 ft.×8 ft. chamber. Three exemplary aerosol collector systems 300 were places on the floor of the chamber. Three 30-min. samples were collected using each of the three systems at a flow rate of 200 L/min. The extraction method used on each of the filters was rotated across each of the three devices to account for any systematic spatial variability in the concentration of aerosolized MS2. Following sampling, the filters from each system were immediately removed and placed in a buffer solution and then subjected to each of the three extraction methods. Extraction fluids comprising MS2 were then analyzed using RT-qPCR to provide the total MS2 viral RNA present in each sample, regardless of the viability of the virus.

For manual extraction, the filter 304 from system 300 was removed using tweezers and inserted into a vial (about 50 ml in volume) having about 5 ml. of extraction fluid. The vial was capped and was shaken vigorously manually for about 30 s. For centrifuge extraction, the filter 304 from system 300 was removed using tweezers and inserted into a vial (about 50 ml in volume) having a plastic insert (see FIG. 5) and having about 5 ml. of extraction fluid. The vial was capped, and the contents were mixed by flipping the vial over several times for about 5 min. The vial was then centrifuged at about 1500 to about 1800 g (about 3000 RPM) for about 5 min. The plastic insert was then removed, and the fluid was analyzed using RT-qPCR. For vortex extraction, the filter 304 was inserted into a vial (about 50 ml in volume) having about 5 ml. of extraction fluid. The vial was capped and vortexed using a vortex mixer/shaker for about 1 min. the fluid was then removed using a pipette for analysis. An exemplary buffer solution in these methods comprised 0.1 mM Tris/HCl pH 7.5, 0.05% Tween-20. FIG. 6B is a box and whisker plot of normalized concentration for each extraction method calculated as the ratio of sample MS concentration to maximum MS2 concentration measured. Manual extraction yielded a higher normalized concentration (0.93) than vortexing (0.80) and centrifuging (0.85). Further, manual extraction produced the most consistent results with a normalized concentration standard deviation of 0.05, compared to that of vortexing (0.09) and centrifuging (0.11).

### Example 4. Concentration Factor for Viruses and Bacillus globigii Spores Using Exemplary System 300 and Extraction Methods

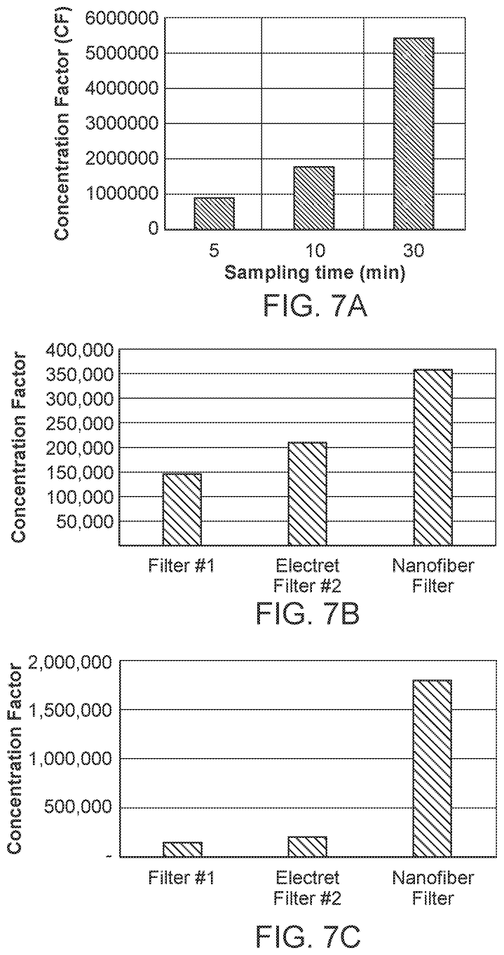

As previously described, the concentration factor (CF) is directly proportional to the particle collection efficiency of the system, the air flow rate pulled through the system and the sampling time and is inversely proportional to the volume of the liquid used to extract the aerosol particles captured using the filter into the liquid (extraction fluid). As shown in FIG. 7A, virus particle concentration factors as high as 500,000 may be realized using exemplary system 300. FIG. 7B shows the CF values measured during collection of Bacillus globigii (Bg) spores using exemplary device 300 using a variety of filters 304. Filter #1 comprised commercially available FLTR face mask material. Filter #2 comprised electret filters supplied by InnovaPrep, LLC (Drexel, Mo.). During these tests, the flow rate of air comprising Bg spores was 200 L/min and the sample was collected for about 10 min. The nanofiber filter comprised a fluoropolymer nanofiber nonwoven mat disposed on a polypropylene backing (FIG. 7B) and a dissolvable filter (FIG. 7C) comprising PVA nanofiber disposed on a polypropylene backing. The filters were then inserted into an exemplary extraction tube with nominal volume of about 50 mL and having about 4 mL of extraction fluid. The extraction fluid comprised between about 0.05% and about 0.08% TWEEN 20 and between about 10 mM (molar) and about 25 mM Tris in hydrochloric acid. Extraction of Bg spores was achieved by simple manual shaking of the extraction tube for about 30 s. As can be seen, CF of about 350,000 was measured using system 300 with the fluropolymer nanofiber filter (FIG. 7B), and about 2×10⁶ with a PVA nanofiber filter (FIG. 7C) that was dissolvable in the extraction fluid.

### Example 5. Viral Titer of Bovine Coronavirus (BCoV) Using Exemplary System 300 and Extraction Methods

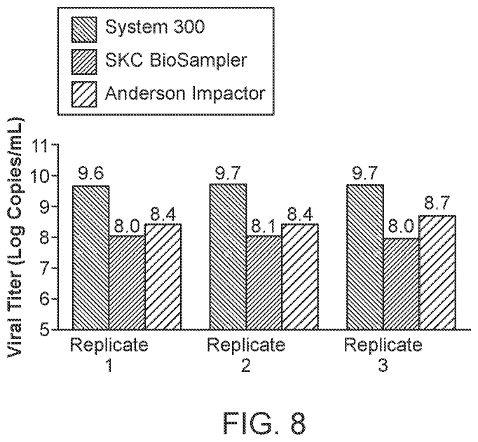

To determine overall collection efficiencies using the exemplary collector systems disclosed herein and to provide a performance comparison with other commercially available aerosol collector systems, a pneumatic nebulizer connected to a wind tunnel was used to aerosolize a high titer suspension of bovine coronavirus (about 10⁷ TCID₅₀/mL, 50% tissue culture infectious dose per mL) to produce an aerosol with a volumetric mean diameter of several micrometers. Aerosol flow velocity profile and particle concentration profiles in the duct were “uniform” in accordance with ASHRAE 52.2 testing criteria. Aerosolized BCoV sampling was carried out using an Andersen cascade impactor (flow rate of 28.3 L/min), an SKC Biosampler (flow rate of 10 L/min) and exemplary system 300 (flow rate of 200 L/min). Exemplary system 300 was adapted to include an inlet port to sample directly inside the tunnel, instead of an open ambient sampling inlet. Sampling was carried out in triplicate using. The filter 304 in system 300 comprised a fluoropolymer nanofiber nonwoven mat disposed on a polypropylene backing. The Andersen impactor and the SKC Biosampler were used as reference collector systems for comparison to exemplary system 300. The wind tunnel was operated at a flow rate of about 50 ft³/min for a sampling time of about 30 min. As a result, the filter in exemplary system 300 was exposed to about 9.93×10⁷ TCID₅₀ of viruses, the Andersen cascade impactor to about 1.41×10⁷ TCID₅₀, and the SKC Biosampler to about 4.97×10⁶ TCID₅₀ during each test. Viability losses during aerosolization or particle deposition in the wind tunnel were disregarded. To recover the captured viruses from the air samplers, a volume of 20 mL cell culture media was used in the SKC Biosampler. Viruses from the Andersen impactor plates were obtained by washing the plates with a cell scraper using 3 ml of BCoV growth media on each stage. In exemplary system 300, the viruses captured on filter 304 were placed into an extraction tube having about 5 mL extraction fluid comprising between about 0.05% and 0.08% TWEEN 20 and between about 10 mM (molar) and about 25 mM Tris in hydrochloric acid and eluted by vigorous shaking. The samples obtained from the three aerosol collector systems were refrigerated immediately after collection and transported to the laboratory for RT-qPCR analysis. About 50 μL of each sample was used for viral RNA extraction with the MagMAX™—96 Viral RNA Isolation kit (Applied Biosystems, Thermo Fisher Scientific, Lithuania) according to the manufacturer's instructions, on a semi-automatic MagMAX Express-96 Deepwell Magnetic Particle Processor (Applied Biosystems, Thermo Fisher Scientific). RNA was eluted with 50 μl of elution buffer and stored at −80° C. until used for viral genome quantification using RT-qPCR protocols. The combined extraction efficiency (TCID₅₀/ml) of BCoV viruses using the three aerosol collector systems is shown in FIG. 8. As can be seen, exemplary aerosol collector system 300 provided virus titers which were higher than the other two devices by 1-2 log orders of magnitude. A similar result was also seen when comparing RT-qPCR concentrations with exemplary system 300 yields virus concentration (copies/ml) of between about 4.23×10⁹ and about 5.3×10⁹. Further, at sample collection times of at least about 10 min., concentration factor (CF) was calculated to be at least 350,000. For sample collection times of about 30 min., CF was calculated to be at least 625,000.

Although the size of a bare virus particle is very small, often as small as 100 nm, the size of exhaled respiratory particles (exhaled breath aerosols, EBA) which may comprise virus particles collected from ambient indoor air are often measured to be in size ranging from about 100 nm to about 5μ. Further, a significant fraction of the aerosol mass is comprised of particles greater than about 2 μm in diameter. The viral particles are typically suspended in aqueous lung fluids that contain water, surfactants, proteins, salts and other chemicals. Particle generation is highest when talking and other activity which causes deep breathing. After these particles are exhaled, they typically shrink on account of water loss. When subsequently measured using exemplary ambient aerosol collector system 300, most of the EBA mass is expected to comprise of particles with size of between about 1 μm and about 5 μm. Filters 104 and 304 used in the exemplary high flow rate aerosol collector systems described herein are highly efficient in capturing particles with size between about 1 μm and about 5 μm, with capture efficiency typically greater than 95% at high flow rates of at least about 200 L/min yielding a collection efficiency of about 80%. Other impact collectors and wet wall cyclone collectors have significantly lower collection efficiencies for particles below about 2 μm even at lower flow rates.

The exemplary methods disclosed herein are most effective when a room's occupants are present in the room during the time the exemplary methods are being implemented. This minimizes the probability that a positive test result is due to fomites or other sources of viral particles (e.g., EBA from individuals that are no longer present). For example, during an 8 h shift at a meat processing facility or in a school classroom, the occupants in the room are both known are usually present in the room during the time the exemplary methods are being implemented. In contrast, a security line at an airport, the occupants in the room would be continuously changing. The exemplary methods are also effective for ACF when the PPV is greater than about 50%, and preferably greater than about 90%. If the identity of the occupants of a room being screened is known, then contact tracing and further testing to identify the spreader may be readily accomplished.

Exemplary systems 100 or 300 may also comprise a CO₂ sensor. CO₂ is an indicator of exhaled breath concentration. Sample collection may be preferentially done in areas inside the indoor space with higher than normal or baseline CO₂ levels measured by the sensor, which are indicative of pockets of exhaled breath. Systems 100 or 300 may be held or positioned at between about 5 ft and 7 ft from the floor to minimize the sampling of particles from fomites which may be kicked up by foot traffic or shed from clothing.

Exemplary sample collector systems 100 or 300 may be configured to determine the size or dimensions of an indoor space using a camera and an “app.” A mobile application software or “app” is a computer program configured to run on a mobile device such as a smart phone, tablet or watch. The mobile device may be operated by the operated of the collector system. The camera may be disposed in the mobile device. Alternately, the camera may be disposed in the collector system. The app may communicate with the collector system using wireless methods such as Bluetooth, WiFi, and the like. The app may be configured to scan the room and estimate the number of people inside the indoor space. To collect a representative air sample in the indoor space that is indicative of EBA produced by people present in the room at a given time, and to guide the operator of the hand portable collector system, sample flow rates, sample collection time, and the areas to be sampled in the indoor space may be determined using the app which may also use the CO₂ levels measured using a CO₂ sensor. Exemplary system 100 or 300 may further comprise a particle counter that may be used to periodically measure the particle count upstream and downstream of the filter and use particle count information to determine parameters that include, but are not limited to, average particle count upstream of the filter to predict preferred sampling time to prevent overloading of the filter average particle count downstream of the filter to predict filter malfunction, and the like.

Exemplary aerosol sample collector system 100 or 300 may be used in conjunction with a wide range of NAAT devices developed for testing nasal and saliva samples. Testing of ambient air samples is not regulated by the U.S. food and Drug Administration (FDA) because air samples are inherently not associated with a specific person or patient. Rather, air samples collected using exemplary system 100 or 300 are environmental samples which can provide valuable information about the safety of the local environment (indoor ambient air) at the time of sample collection. When the sample is analyzed using a suitable NAAT device, an analysis result may be obtained in less than about 30 min. from the time of starting sample collection using exemplary device 300, a positive result may be used as a basis to move people in that indoor space outdoors and take other corrective action. For example, under the direction of a health care professional, a rapid test (e.g., using Abbot Laboratories' BinaxNOW antigen COVID-19 self-test kit) may be performed on each person who were present the room at the time the ambient aerosol sample was collected.

The exemplary PON systems and methods described herein may be used to collect samples from school buses used for transporting students to and from school. Buses are also used to transport school band members, sports and other academic teams, cheerleaders, and in some cases, parents to competitions. Any activity that creates a congregation, for example, including but not limited to, religious and other ceremonies, a classroom, locker room, gymnasium, break room, cafeteria, and theater may be considered to a suitable indoor areas for testing for the presence of airborne virus particles such the SARS-CoV-2 virus using the exemplary systems and methods.

The Abstract is provided to comply with 37 C.F.R. § 1.72(b), to allow the reader to determine quickly from a cursory inspection the nature and gist of the technical disclosure. It should not be used to interpret or limit the scope or meaning of the claims.

Although the present disclosure has been described in connection with the preferred form of practicing it, those of ordinary skill in the art will understand that many modifications can be made thereto without departing from the spirit of the present disclosure. Accordingly, it is not intended that the scope of the disclosure in any way be limited by the above description.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method and apparatus modes.

Further, each of the various elements of the disclosure and claims may also be achieved in a variety of manners. This disclosure should be understood to encompass each such variation, be it a variation of an implementation of any apparatus implementation, a method or process implementation, or even merely a variation of any element of these.

Particularly, it should be understood that the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this disclosure is entitled. It should be understood that all actions may be expressed as a means for taking that action or as an element which causes that action. Similarly, each physical element disclosed should be understood to encompass a disclosure of the action which that physical element facilitates.

In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Further, the use of the transitional phrase “comprising” is used to maintain the “open-end” claims herein, according to traditional claim interpretation. Thus, unless the context requires otherwise, it should be understood that variations such as “comprises” or “comprising,” are intended to imply the inclusion of a stated element or step or group of elements or steps, but not the exclusion of any other element or step or group of elements or steps. Such terms should be interpreted in their most expansive forms so as to afford the applicant the broadest coverage legally permissible.

What is claimed is:

1. A method for active case finding in an indoor space for a respiratory disease associated with a group of people who are present in the indoor space, the method comprising:
collecting an aerosol sample comprising exhaled breath aerosols (EBA) from ambient air in the indoor space onto a filter substrate using a high flow rate hand portable aerosol sample collector system;
extracting aerosol particles from the filter substrate on-site into a liquid sample; and
analyzing the liquid sample using a nucleic acid amplification test (NAAT) analysis system on-site to confirm or eliminate the presence of the respiratory pathogen in the indoor space.

2. The method of claim 1 wherein the analyzing step comprises analyzing the sample using technologies that comprise at least one of PCR, RT-PCR, isothermal nucleic acid amplification, and ELISA.

3. The method of claim 1 wherein the analyzing step comprises analyzing the sample using isothermal nucleic acid amplification.

4. The method of claim 1 wherein the method is characterized by a concentration factor (CF) of at least 350,000.

5. The method of claim 1 wherein the collecting step further comprises moving the hand portable aerosol sample collector system proximate to the group of people during the collecting step.

6. The method of claim 1 further comprising the step of isolating and diagnosing each member of the group for the respiratory disease if the sample analysis confirms the presence of a respiratory pathogen by indicating a positive test result associated with the aerosol sample.

7. The method of claim 1 wherein the high flowrate aerosol sample collector system is configured to move air at a flow rate of at least about 200 L/min through the filter substrate.

8. The method of claim 1 wherein the aerosol sample collection time using the aerosol sample collector system is between about 10 min and 30 min.

9. The method of claim 1 wherein the time to obtain a diagnostic test result is less than about 60 min. measured from the start of the aerosol sample collector system.

10. The method of claim 1 wherein the time to obtain a diagnostic test result is less than about 30 min. measured from the start of the aerosol sample collector system.

11. The method of claim 1 wherein the on-site analysis system is characterized by a positive predictive value of at least 50%.

12. The method of claim 1 further comprising the step of pooling multiple aerosol samples into one combined aqueous sample prior to analyzing using the on-site analysis system.

13. The method of claim 1 wherein the extracting step comprises extracting aerosol particles from the filter substrate using an extraction fluid comprising between about 0.05% and about 0.08% TWEEN 20 and between about 10 mM (molar) and about 25 mM Tris in hydrochloric acid.

14. The method of claim 1 wherein the extracting step comprises extracting aerosol particles from the filter substrate using an extraction fluid characterized by a pH of between about 7.5 and about 8.

15. The method of claim 1 wherein the extracting step is completed in less than about 5 min.

16. The method of claim 1 wherein the extracting step is completed in less than about 2 min.

17. The method of claim 1 wherein the extracting step comprises:
removing the filter from the aerosol sample collector system;
positioning the filter inside a tube having a predetermined volume of extraction fluid and capping the tube; and
shaking the tube vigorously for 30 s.

18. The method of claim 17 wherein the volume of extraction fluid in the capped tube is less than about 5 ml.

19. The method of claim 17 wherein the volume of extraction fluid in the capped tube is less than about 1 ml.

20. The method of claim 17 wherein the volume of extraction fluid in the capped tube is between about 4 ml and about 8 ml.

21. A hand portable aerosol sample collector system comprising:
a filter holder to support a filter substrate;
a fan to pull ambient air comprising aerosol particles through the filter substrate at a flow rate selectable by an operator and for a sampling time selectable by an operator;
and a housing to substantially enclose the filter holder and the fan wherein the system is configured to operate at a noise level of less than about 70 dB.

22. The system of claim 21 wherein the fan is configured to pull air through the filter at a flow of between about 200 L/min and about 500 L/min.

23. The system of claim 21 wherein the system is powered by a rechargeable battery pack.

24. The system of claim 21 wherein the filter substrate comprises at least one of a polyester felt, electret filters, a fluoropolymer nanofiber nonwoven mat disposed on a backing material, and a combination thereof.

25. The system of claim 24 wherein the backing material comprises at least one of cellulose acetate, nylon, and polypropylene.

26. The system of claim 21 wherein the filter substrate is coated with a water-soluble coating.

27. A sample extraction kit for extracting aerosol particles from a filter disposed in a high flow rate aerosol sample collector system, the extraction kit comprising:
a pair of tweezers; and,
a capped tube having a predetermined volume of extraction fluid.

28. The extraction kit of claim 27 wherein the volume of the capped tube is between about 25 ml and about 50 ml.

29. The extraction kit of claim 27 wherein the volume of the extraction fluid is about 4 ml.

30. The extraction kit of claim 27 wherein the volume of the extraction fluid is between about 1 ml and about 6 ml.

31. The extraction kit of claim 27 wherein the extraction fluid comprises between about 0.05% and about 0.08% TWEEN 20 and between about 10 mM (molar) Tris and 25 mM in hydrochloric acid.

32. The extraction kit if claim 27 wherein the extraction fluid comprises about 0.05% TWEEN 20 and about 10 mM (molar) Tris in hydrochloric acid.

33. The extraction kit of claim 27 wherein the kit is disposable.

34. The extraction kit of claim 27 wherein the kit has a unique bar code for identifying the kit.

35. A sample extraction kit for extracting aerosol particles from a filter substrate having aerosol particles collected using a high flow rate aerosol sample collector system, the extraction kit comprising:
a capped centrifuge tube having a predetermined volume of extraction fluid; and,
an insert component configured to receive the filter substrate and to be slidably disposed inside the centrifuge tube wherein the insert has a substantially open bottom end that allows the extraction fluid to enter the insert component.

36. A hand portable aerosol sample collector system comprising:
a filter holder to support at least one filter substrate;
a fan to pull ambient air comprising aerosol particles through the filter substrate at a flow rate selectable by an operator and for a sampling time selectable by an operator;
and a housing to substantially enclose the filter holder and the fan wherein the system is configured to operate at a noise level of less than about 70 dB.

37. The system of claim 36 wherein the filter substrate is between about 2 in. and about 3 in. in diameter.

38. The system of claim 36 wherein the filter substrate comprises at least one of a polyester felt, electret filters, and a fluoropolymer nanofiber nonwoven mat disposed on a backing material, and a combination thereof.

39. The system of claim 38 wherein the backing material comprises at least one of cellulose acetate, polypropylene, and nylon.

40. The system of claim 36 wherein the filter substrate comprises polyvinyl acetate nanofiber disposed on a backing material.

41. The system of claim 40 wherein the backing material comprises at least one of cellulose acetate, polypropylene, and nylon.
